(19) United States
(12) Patent Application Publication
(10) Pub. No.: US 2021/0373028 A1
(43) Pub. Date: Dec. 2, 2021

(54) NOVEL FLUORESCENCE DYES AND NOVEL ANALYTICAL PROCESSES FOR GLYCAN ANALYSIS

(71) Applicant: ABBERIOR GMBH, Göttingen (DE)

(72) Inventors: Daniel BERNDT, Bielstein (DE); Shamil NIZAMOV, Göttingen (DE); Lars KASTRUP, Göttingen (DE)

(21) Appl. No.: 17/281,679

(22) PCT Filed: Sep. 25, 2019
(86) PCT No.: PCT/EP2019/075940

§ 371 Date: Mar. 31, 2021

(30) Foreign Application Priority Data
Oct. 1, 2018 (DE) 10 2018 124 199.2

(51) Int. Cl. G01N 33/58; C09B 15/00; C09B 69/10

(52) U.S. Cl. CPC G01N 33/582; C09B 69/109; C09B 15/00

(57) ABSTRACT

The invention relates to a fluorescent marker according to the following formula I

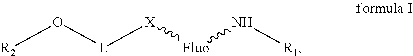

wherein Fluo is selected from the group consisting of substituted or non-substituted C10-C30 acridone, aminoacridine or pyrene, L is a linker and the moiety X is either a —SO₂— or a —PO(OH)— moiety. Furthermore, the present invention relates to the use of the fluorescent markers of the invention, in particular also as a standard in glycan analysis, conjugates of fluorescent markers and glycans, as well as a kit-of-parts containing the fluorescent markers of the invention.

### CROSS-REFERENCE TO RELATED APPLICATIONS

This application is a U.S. National Phase Application under 35 U.S.C. 371 of International Application No. PCT/EP2019/075940, filed on Sep. 25, 2019, which claims the benefit of German Patent Application No. 10 2018 124 199.2, filed on Oct. 1, 2018. The entire disclosures of the above applications are incorporated herein by reference.

### FIELD

The invention relates to a fluorescent marker according to the following formula I

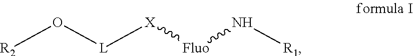

wherein Fluo is selected from the group consisting of substituted or non-substituted C10-C30 acridone, aminoacridine or pyrene, L is a linker and the moiety X is either a —SO₂— or a —PO(OH)— moiety. Furthermore, the present invention relates to the use of the fluorescent markers of the invention, in particular also as a standard in glycan analysis, conjugates of fluorescent markers and glycans, as well as a kit-of-parts containing the fluorescent markers of the invention.

### BACKGROUND

This section provides background information related to the present disclosure which is not necessarily prior art.

The investigation of biological macromolecules is still an analytical challenge today. On the one hand, the samples to be analyzed are usually more or less inhomogeneous mixtures whose physical and chemical properties depend not only on the chemical composition as such but also on other factors, such as the 3-dimensional structure of the molecules. On the other hand, significant interactions can also occur between the individual components, which represents a further complication in the analysis and in the interpretation of the results obtained.

The above applies in particular to glycan analysis. Glycans are generally defined as polymeric compounds consisting of a large number of monosaccharide building blocks linked to each other by a glycosidic bond. Glycoconjugates, for example glycoproteins or glycolipids, which are frequently found in eukaryotes and less frequently in prokaryotes, also fall into this class. These compounds perform a variety of different tasks in living organisms. These include, for example, the structural integrity of cell membranes, the formation of the extracellular matrix, signal transduction, protein folding and intercellular information exchange. Based on the variety of structural designs and the multitude of biological functions, glycan analysis has become increasingly important for the pharmaceutical and food industries in recent years.

The molar mass of glycans and their conjugates can be determined, for example, by capillary electrophoresis (CE), which allows rapid and efficient separation of even the smallest sample volumes on the basis of molecular charge. A variation of the CE principle is followed in capillary gel electrophoresis (CGE). In this method, the capillaries are filled with a gel former in addition to the electrolyte. Based on the gel component, the separation is also significantly enhanced by the size properties (molecular sieve effect) of the molecules to be investigated, since larger molecules experience a significantly higher migration resistance through the gel network than smaller ones. The use of the polymer gel thus allows a significant spreading of the physical influencing factors, which in this method is a function of the molecular shape in addition to the charge. The latter considerably expands the range of applications and even enables the separation of macromolecular structural isomers.

For the detection of glycans in CE and CGE, the use of laser-induced fluorescence spectroscopy (LIF) has become increasingly common. Due to the fact that glycans usually do not have fluorophores, additional fluorophores have to be introduced into the target molecule to study the substances. The latter is due in particular to the fact that the optical path length within the capillaries is very short and the samples have only a very small amount of analytes, so that very high sensitivity requirements must be placed on the detection methods. The boundary conditions therefore require the use of highly efficient fluorophores and highly efficient optical components. A “common” setup is based on an argon laser with fluorescence excitation wavelengths at 488 nm and 514 nm and a photodetector with several detection windows.

For the determination of absolute molecular weight values, the systems used must be calibrated by the use of reference substances. Fluorophore-labeled DNA fragments (“DNA ladders”) with 35-500 nucleotides are frequently used as standards. The reference substances are added to the capillaries either before or after the actual sample. This has the advantage that sample/reference can work with the same fluorophores. With the DNA references, uniform elution distances can be provided over a very wide elution range. However, a disadvantage is that the basic structure of polynucleotides differs from that of glycans. For this reason, analogous to the DNA ladder, dextran ladders are also offered as reference substances. Compared to glycans, these have structurally similar monomers and therefore show much more comparable migration behavior. A disadvantage, however, is that there are only a few suitable fluorophores that satisfy the analytical constraints of the method. This is further complicated by the fact that they also exhibit very similar spectral properties.

The use of fluorescent labels in analytics is also addressed in the patent literature. For example, EP 0 943 918 A1 describes low molecular weight fluorescent labeling complexes with large wavelength shifts between the absorption of one dye in the complex and the emission of another dye in the complex. These complexes can be used, for example, for multiparameter fluorescence cell analysis using a single excitation wavelength. The low molecular weight of the complex allows materials labeled with the complex to penetrate cell structures for use as probes. The labeling complexes are synthesized by covalent bonding through linkers to form donor-acceptor complexes. Resonance energy transfer from an excited donor to a fluorescent acceptor provides wavelength shifts of up to 300 nm. The fluorescent labeling complexes preferably contain reactive groups for labeling functional groups to target compounds, such as derivatized oxy- and deoxynucleic acids, antibodies, enzymes, lipids, carbohydrates, proteins, and other materials. The complexes may contain functional groups that enable covalent bonding to materials bearing reactive groups.

Furthermore, WO 2009 078 970 A1 discloses fluorescent dyes in general according to the following formula

F—Y=Ψ

In addition, the document provides a wide range of fluorescent dyes and kits containing them that are applicable for labeling a variety of biomolecules, cells, and microorganisms. The document also provides various methods for using the fluorescent dyes for research and development, forensic identification, environmental studies, diagnosis, prognosis and/or treatment of disease states.

Despite the fluorescent markers already known in the field of analytics, there is still an interest in new compounds that are able to deliver highly accurate and reproducible results even under difficult boundary conditions of the respective analytical method.

### SUMMARY

This section provides a general summary of the disclosure, and is not a comprehensive disclosure of its full scope or all of its features.

This task is fulfilled by the fluorescent markers described in the independent claims, the fluorescent marker-glycan conjugates and the kit-of-parts according to the invention. Preferred embodiments thereof are set forth in the dependent claims.

According to the invention, a fluorescent label according to the following formula I

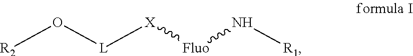

wherein

### R₁=—H or C1-C6 Alkyl;

R₂=—H or —PO(OH)₂

### X=—SO₂— or —PO(OH)—;

L=—(CH₂)ₙ—CHR₃—CH₂—, n=1, 2, 3 and R₃=—H or —(CH₂)—OPO(OH)₂;
wherein Fluo is selected from the group consisting of substituted or non-substituted C10-C30 acridones, aminoacridines or pyrenes.

It has been found that the fluorescent markers of the invention have a large number of positive properties in the field of analysis and, in particular, in the field of capillary electrophoretic investigation of glycans. Due to their structure, the fluorescent markers are able to be easily and reproducibly coupled to glycans via reductive amination. This is most likely due to the amino group present, which has a pKₐ value of about 4-5. The other structural components of the fluorescent label are stable under usual coupling conditions, so that a reliable and highly efficient coupling can be obtained. The fluorescent labels can exhibit a high net negative charge and are thus particularly suitable for rapid separations in common electrophoresis buffers, for example at a pH around 8. Furthermore, the claimed structures exhibit high absorption in the commonly used wavelength ranges of Ar-lasers around 488 nm. As a result, these fluorescent markers can be used with standard gel electrophoresis equipment. The fluorescent markers are also characterized by high extinction coefficients and excellent quantum yields. Particularly noteworthy is that the emission of the claimed fluorescent markers lies outside the emission range of fluorescent markers known from the state of the art. This makes it possible for the first time that reference and sample can be marked with different fluorescence markers and measured at the same time. The signals of both components, reference and sample, can be easily distinguished by the different emission wavelengths. The emission wavelengths of the structures claimed here lie in a range around 560-600 nm, while the fluorescent markers used in the prior art provide emission around 500 nm. The emission maxima are therefore far enough away to allow significant assignment. Another advantage is that the fluorescence properties of the claimed fluorophores do not change at all or only marginally by coupling to glycans. In sum, fluorescence markers were obtained which, due to the existing boundary conditions, are extremely advantageous for state-of-the-art CE and GCE methods.

The fluorescent marker according to the invention have a specific structure according to the formula I

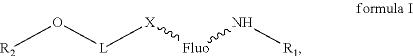

This means that the fluorescent labels have at least one fluorophore unit Fluo, which is covalently bound to a functional group X as well as directly covalently bound to a nitrogen-containing group. The functional group X is in turn covalently bonded to a linker group L, which is oxygen bridged to the terminal group R₂.

According to the invention, the amino group R1 can be a primary (—H) or a secondary amine, which then carries a C1-C6 alkyl group. Preferably, this group can be used as a binding site for coupling glycans. Despite the proximity to the fluorophore, it has been shown that even after coupling of large glycan residues to the amino group in this position, the spectroscopic properties, and here in particular the position of the emission maximum, change only insignificantly. This can reduce the equipment required for the measurement and provides very reproducible measured values.

The terminal group R₂ can be hydrogen or —PO(OH)₂. Thus, according to the invention, phosphoric acid derivatives or alcohols are obtained at this position. As a function of the buffer system, this group can of course also be charged, for example as an alcoholate or phosphate. By this embodiment, further charges can be generated at the fluorescent marker, which can increase the migration speed of conjugates in the context of CE.

The bridging group X can be either —SO₂— or —PO(OH)—. These groups have been found to be particularly efficient for changing the emission wavelength of the fluorescent label. By using these groups, one is able to shift the emission wavelength towards longer wavelengths, and fluorophores with high quantum yields are obtained via these substituents. Moreover, by introducing this group, the conversion with glycans to conjugates is not negatively affected. Similar reaction rates are obtained without significant side reactions.

The linker L can be a pure hydrocarbon (R₃=—H) or can also carry another group that can be ionized as a function of pH (R₃=—(CH₂)—OPO(OH)₂). The latter may be particularly preferred if higher charge densities on the fluorescent marker are desired.

The group Fluo can be selected from the group consisting of substituted or unsubstituted C10-C30 acridones, aminoacridines or pyrenes. This means that the fluorophore main body, which is bound to the amino group and via the functional group X to the linker, belongs to one of the 3 mentioned compound classes. Surprisingly, it was found that these 3 main bodies in particular are suitable to provide fluorescence labels by the structure according to the invention, which have a specific emission wavelength range. This is all the more surprising since these three Fluo main bodies can have a very different structure after all. Without being bound by theory, the three different main bodies in the context of the structure according to the invention exhibit very comparable electrical properties, resulting in similar absorption and emission behavior. Moreover, the chemical stability of these groups is comparable, so that efficient coupling reactions with glycans can be obtained with very comparable kinetics and with similar selectivity. It is further surprising that these groups show comparable invariance with respect to the shift of the emission maximum after coupling. This may be due to the fact that the different Fluo main bodies have a similar spatial structure in addition to comparable electronic properties.

According to the invention, the group of C10-C30 acridones includes the following example compounds;

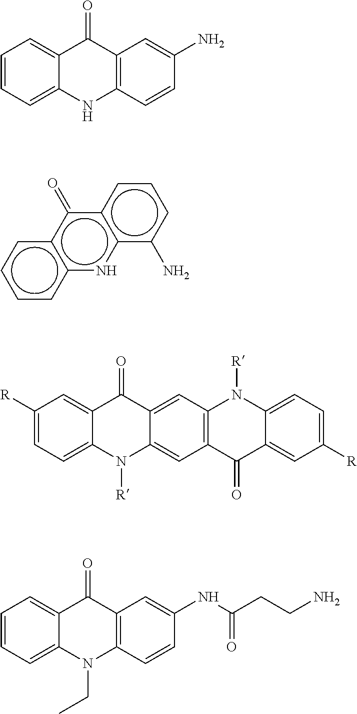

According to the invention, the group of C10-C30 aminoacridines includes the following example compounds:

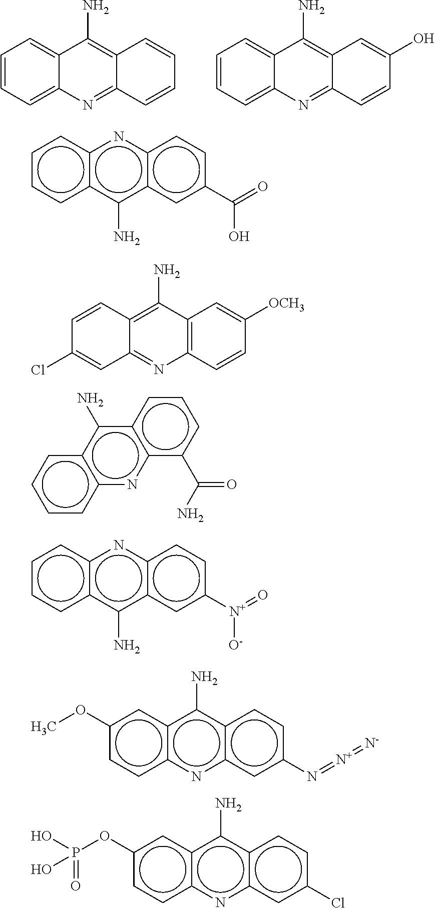

According to the invention, the following compound falls into the group of C10-C30 pyrenes:

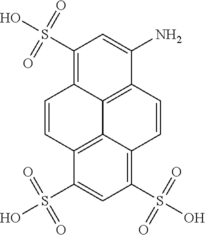 [SMILES: Nc1cc(S(=O)(=O)O)c2ccc3c(S(=O)(=O)O)cc(S(=O)(=O)O)c4ccc1c2c43]

In addition, these compounds may be substituted at each binding site by further functional groups such as —OH, —COOH, OR, —NH₂, —NHR, —NR₂ with R alkyl, halogens, —SO₃H, —OPO₃H₂ or may already comprise a part or all of the groups X and NH—R₁.

In a preferred embodiment of the fluorescent marker, fluo may be selected from the group consisting of the fluorophores according to formulae II-IV

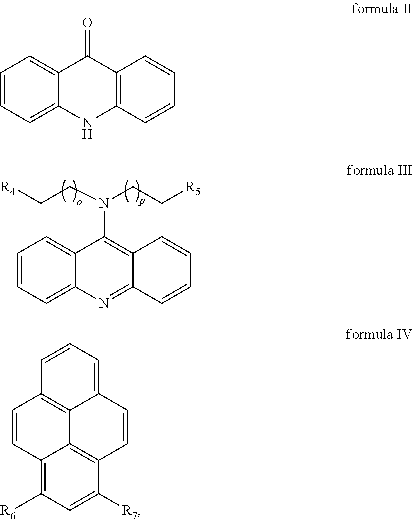

wherein R₄, R₅=each independently of one another —OPO(OH)₂ or —OH; R₆, R₇=each independently of one another —SO₂—CH₂—CHR₂—(CH₂)_{q}—R₄ or —PO(OH)—CH₂—CHR₂—(CH₂)_{q}—R₄ and q=1, 2, 3; o, p=each independently of one another 1, 2, 3; and the Fluo according to the formula II-IV can be linked at each possible binding site to NH and X. These structures from the different classes have proven to be particularly suitable as fluorescent markers in glycan analysis. In addition to what has already been said about the different groups, the comparable molecular weight, size and electronic properties seem to give rise to very similar fluorescent marker properties. These compounds are stable under CE conditions and can be copolymerized to glycans within very similar reaction times. The conjugates obtained are chemically stable and show excellent shelf-lives even in the presence of an aqueous buffer. The compounds are also compatible with common buffer systems in CE and can cover a broad charge spectrum as a function of pH. The linkage to the group X and the amino group can occur at any position of the fluorophore backbone. These can preferably be the aromatic structures of the fluorophore. These binding sites have proven to be particularly stable.

In a preferred embodiment of the fluorescent marker, the fluorophores Fluo may correspond to formulas V-VII

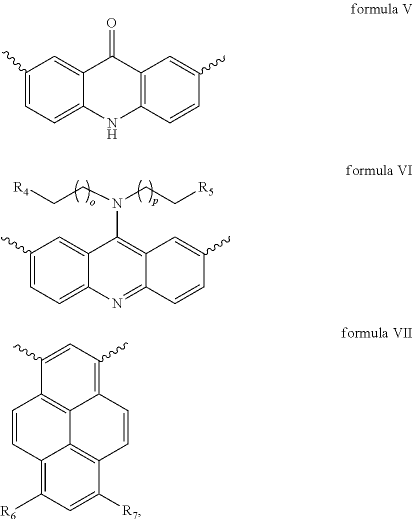

wherein the fluorophore can be linked via the curved bonds to NH and X. The definition of the further Markush groups is defined above. These structures with the specific binding sites to the further functional components of the fluorescent marker have proven to be particularly suitable. Without being bound by theory, these specific binding sites result in steric configurations of the fluorescence marker that allow a particularly simple and efficient interaction with glycan macromolecules. As a result, the coupling reactions can be carried out quickly and efficiently. Stable conjugates are obtained within short reaction times, which are also very stable in aqueous systems.

In the context of a further characteristic of the fluorescence marker, X can be —SO₂—. In particular, fluorescent labels with the bridging unit shown have proven to be particularly suitable for fluorescence analysis. The bridging SO₂-unit seems to be responsible for the strong shift in fluorescence emission and provides a distinctly different emission behavior compared to state-of-the-art fluorescence markers. This is one of the bases that one can operate two different fluorescence markers with different emission windows within one measurement. This unit also provides a chemically very stable molecule, which remains stable even under the harsh coupling conditions to glycans.

Within a preferred embodiment of the fluorescence marker, in the linker L R₃=—(CH₂)—OPO(OH)₂. The basic form of the fluorescence marker according to the invention is so flexible that even by linking further functional subgroups, as shown above, the basic fluorescence properties change only insignificantly. In this way, it is possible to increase the net charge of the fluorescent marker via additional de-protonatable groups, which can contribute to faster and more specific separation in the field of glycan analysis. Via the additional functional groups with a phosphorus central atom, these fluorescent markers can also be used flexibly with different solvents at different pH values. This can contribute to a significant flexibilization in glycan analysis.

Within a preferred aspect of the fluorescent label, R₄ and R₅ may each be —OPO(OH)₂ in the fluorophore according to formula III or VI. The basic form of the fluorescent label according to the invention is so flexible that even the addition of further functional subgroups in structures II and VI, as shown above, changes the basic fluorescence properties only insignificantly. In this way, one is able to increase the net solubility of the fluorescent label via further de-protonatable groups, which can contribute to a faster and more specific separation in the field of glycan analysis. Via the additional functional groups with a phosphorus central atom, these fluorescent markers can also be flexibly applied with different solvents at different pH values. This can contribute to a significant increase in flexibility in glycan analysis.

According to a preferred embodiment of the fluorescent marker, in the fluorophore according to formulae IV and VII, R₆ and R₇ can each be —PO(OH)—CH₂—CHR₂—(CH₂)—OPO(OH)₂. The basic form of the fluorescent marker according to the invention is so flexible that even by attaching further functional subgroups in structures IV and VII, as shown above, the basic fluorescence properties change only insignificantly. In this way, one is able to increase the net charge of the fluorescent label via further de-protonatable groups, which can contribute to a faster and more specific separation in the field of glycan analysis. Via the additional functional groups with a phosphorus central atom, these fluorescent markers can also be applied flexibly with different solvents at different pH values. This can contribute to a significant increase in flexibility in glycan analysis.

Furthermore, the use of the fluorescent markers according to the invention as a standard in a glycan analysis is according to the invention. The fluorescent markers of the invention are particularly suitable as a standard for the analysis of glycans. Based on the structure of the fluorescent markers, covalent binding sites to glycans are obtained, which can be easily and reproducibly reacted with functional groups of the glycans in the course of controlled reactions. In this way, glycans or, in general, sugars with a specific molecular weight can be easily coupled to the fluorescent labels of the invention. Thus, labeled glycan samples with specific molecular weight and reproducible migration properties are obtained, which can also be used as MW reference samples in CE or GCE. Due to their comparable chemical structure, these molecules are much more suitable compared to DNA ladders. A further advantage results from the changed emission behavior and here in particular the changed emission wavelength. This means that both the sample and the reference can be detected simultaneously in different emission windows within one separation, provided that the sample is labeled with a state-of-the-art fluorescence marker. Since sample and reference are separated under exactly the same conditions, these measurements are more reproducible than measurements in which the reference is examined with the same fluorescent label in a pre- or post-experiment. The use of the fluorescent markers according to the invention can therefore save analysis time and costs. Usefully, the glycan standards used can be standards as a function of the molecular weight of the glycans.

Further according to the invention is a conjugate of a fluorescent marker and a glycan, wherein the glycan is covalently bound to at least one fluorescent marker according to the invention. Due to the chemical structure, the fluorescence markers according to the invention can be coupled efficiently to glycans. In this way, one or more fluorescence markers can be coupled to the glycans, thus making them accessible to fluorescence examination. Advantageously, the conjugates can carry a larger number of charges as a function of the pH value present. This can be achieved, for example, by deprotonation of the acidic groups of the fluorescent label. The conjugates can thus carry a higher net charge and electrophoretic measurements in particular can be carried out more quickly.

In a preferred variant of the conjugate, the glycan may be selected from the group consisting of mono-, oligo-, polysaccharides, glycoproteins, glycolipids, proteoglycans or mixtures thereof. The above-mentioned group of glycans can be coupled rapidly to the fluorescent labels of the invention without the formation of undesirable by-products. In this way, samples from the group of glycans mentioned above as well as, for example, molecular weight standards can be measured within one experiment. The advantages already discussed for the fluorescent marker are obtained.

Further according to the invention is a kit-of-parts comprising at least one or more containers with one or more solvents and one or more containers each with the fluorescent markers or conjugates according to the invention. In particular, a sales package containing one or more glycan-fluorescent marker conjugates, preferably with different molecular weights, can significantly facilitate the fluorescence examination of unknown glycan samples. The conjugates may already be present in solution, for example in a buffer, or as an isolated substance. The latter can then, for example, be processed together with a buffer provided in the kit to form a conjugate solution shortly before the measurement. This can be advantageous because the amount to be used can be adjusted by the mixing ratio of buffer to conjugate. In addition, the amount can be adapted to the sensitivity of the experimental setup. Preferably, the kit can have one, more preferably two or three conjugates with different molecular weights. The use of several conjugates can contribute to a more reliable measurement via a possible further ma-thematic processing of the information obtained. However, it is also possible to provide a kit for staining glycans.

In a preferred embodiment of the kit, the kit may be a “labeling” kit for use in labeling glycans. In addition to the dye according to the invention as an optional component, this labeling kit can also comprise other optional components such as one or more buffer solutions, one or more reducing agents (e.g. sodium cyanoborohydride, 2-picoline borane), consumables for carrying out the staining (vessels, etc.) and staining instructions. By means of this kit, glycan samples can be stained according to the invention.

In a further embodiment of the kit, the kit may be a glycan ladder kit for use as an internal standard. The kit may optionally comprise a mixture of stained glycans, preferably dextrans, of different molecular weights and preferably in lyophilized form. Furthermore, the kit may optionally include solvents or buffers, consumables for staining (vessels, etc.) and instructions for reconstitution and use. By means of this kit, labeled glycans with known molecular weights can be provided as internal standards in the investigation of glycans with unknown molecular weights.

Further areas of applicability will become apparent from the description provided herein. The description and specific examples in this summary are intended for purposes of illustration only and are not intended to limit the scope of the present disclosure.

### DRAWINGS

Further advantages and advantageous embodiments of the objects according to the invention are illustrated by the figures and explained below. It should be noted that the figures are descriptive only and are not intended to limit the invention in any way.

The figures show:

FIG. 1 A synthetic route for a phosphorylated acridone fluorescent marker according to compound 1;

FIG. 2 Absorption/emission spectra of example compound 1 (in 0.05 M TEAB buffer with excitation at 488 nm) as an isolated fluorescent label (-) and as a glucose conjugate ( - - - ) in the wavelength range between 250 and 800 nm.

### DETAILED DESCRIPTION

Example embodiments will now be described more fully with reference to the accompanying drawings.

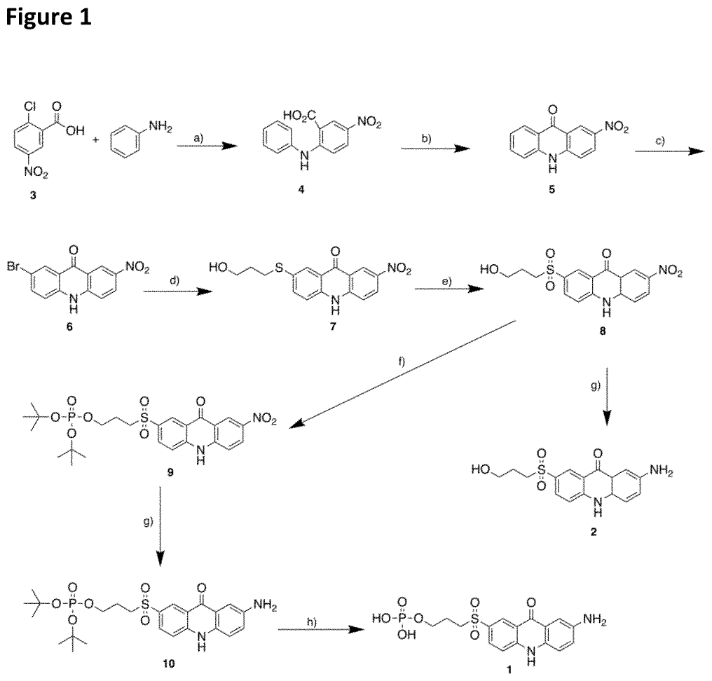

FIG. 1 shows a possible synthetic route for obtaining compound 1. The individual synthesis steps and reaction conditions are described further below in the example section.

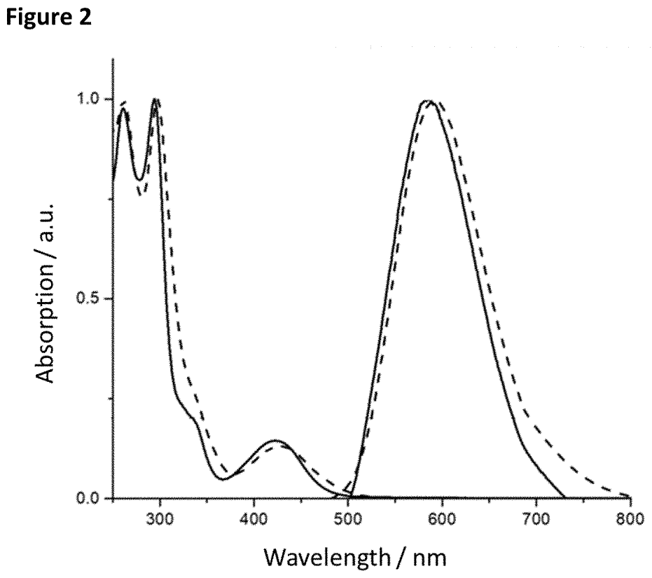

FIG. 2 shows the absorption and emission properties of a fluorescent label according to the invention and of a conjugate of a fluorescent label and a simple sugar. Shown are the normalized absorption and emission spectra of 7-[(3-phosphopropyl)sulfonyl]-2-amino-acridin-9(10H)-one (1) and its glucose conjugate. The absorbance characteristics were measured in 0.05 M TEAB buffer (triethylammonium bicarbonate buffer) at pH 8.0. Excitation was performed at 488 nm. Fluorescence emission can be clearly seen with a maximum around 585 nm. Due to the formation of the conjugate, the emission maximum is only slightly shifted to higher wavelengths and lies at approx. 590 nm. The spectroscopic properties of the fluorescent marker make it particularly suitable for glycan analysis. The fluorescent marker can be used alone or, due to its emission maximum, simultaneously in combination with a prior art fluorescent marker. Due to the different emission windows, two fluorescence markers can be detected independently of each other.

### Examples

Synthesis of the Example Compounds

A possible synthesis route to obtain the compound (1) is shown in FIG. 1.

### (I) 5-Nitro-N-phenyl-anthranilic acid (4)

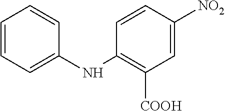 [SMILES: O=C(O)c1cc([N+](=O)[O-])ccc1Nc1ccccc1]

2-chloro-5-nitrobenzoic acid (7.5 g, 38 mmol) are dissolved in 50 mL n-butanol at 40° C. Ani-line (7.7 mL, 83 mmol, 2.2 eq.) and DIEA (82.5 mmol, 14.4 mL, 2.2 eq.) are added. The mixture is stirred under reflux (120° C.) for 3 days. After cooling to room temperature, the volatile components are evaporated off in vacuo. A brown oil is obtained. The residue is transferred to a vigorously stirred mixture of crushed ice and hydrochloric acid (1 M, 100 mL). The precipitated product is filtered off, washed with water and dried in air. The crude product is recrystallized in acetic acid (180 mL), filtered off and washed with diethyl ether. After drying in vacuo, compound (4) (6.37 g, 24.7 mmol, 66%) is obtained as a yellow residue.

MS (ESI): m/z (positive mode)=259.1 [M+H]⁺, 281.1 [M+Na]⁺.

HR-MS (ESI): C13H10N2O4: Calc. [M+H]+ 259.0713, found 259.0714, [M+Na]+: Calc. 281.0533, found: 281.0535.

¹H-NMR (400 MHz, DMF-d₇): δ [ppm]=14.42 (s, 1H, COOH), 10.53 (s, 1H, NH), 8.87 (d, J=2.8 Hz, 1H, 6-H), 8.25 (dd, J=9.4, 2.8 Hz, 1H, 4-H), 7.56-7.49 (m, 2H, 3′-H), 7.47-7.42 (m, 2H, 2′-H), 7.32 (m, 1H, 4′-H), 7.26 (d, J=9.4 Hz, 1H, 3-H),

¹³C-NMR (101 MHz, DMF-d₇): δ [ppm]=170.2, 154.0, 139.7, 138.2, 131.0, 130.4, 129.7, 126.9, 125.2, 114.4, 112.3,

TLC: R_{f}=0.54 (MeOH/H₂O, 80/20)

### (II) 2-Nitroacridin-9(10H)-one (5)

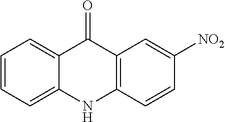 [SMILES: O=c1c2ccccc2[nH]c2ccc([N+](=O)[O-])cc12]

Compound 4 (0.51 g, 1.96 mmol) is dissolved in POCl₃ (5 mL) and the reaction mixture is stirred under reflux (125° C.) for 3 hours. The POCl₃ is evaporated in vacuo and crushed ice (50 g) and hydrochloric acid (1 M, 100 mL) are added to the residue. The mixture is boiled under reflux for one hour until a yellow product precipitates. The precipitate is filtered, washed with water, cold methanol and diethyl ether and then dried in air. A pure 2-nitroacridin-9(10H)-one (0.40 g, 1.66 mmol, 85%) is obtained as a light yellow solid.

¹H-NMR (400 MHz, DMSO-d₆): δ [ppm]=12.37 (s, 1H, NH), 8.99 (d, J=2.7 Hz, 1H, 1-H), 8.49 (dd, J=9.2, 2.7 Hz, 1H, 3-H), 8.26 (dd, J=8.1, 1.5 Hz, 1H, 8-H), 7.84 (ddd, J=8.4, 7.0 Hz, 1H, 7-H), 7.69 (d, J=9.2 Hz, 1H, 4-H), 7.61 (dd, J=8.4, 1.5 Hz, 1H, 5-H), 7.39 (ddd, J=8.1, 7.0 Hz, 1H, 6-H),

HPLC: t_{R}=9.87 min (ACN/H₂O+0.1% TFA; 30/70→10/0 in 12 min; detection at 254 nm)

### (III) 7-Bromo-2-nitroacridin-9(10H)-one (6)

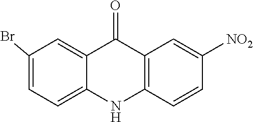 [SMILES: O=c1c2cc(Br)ccc2[nH]c2ccc([N+](=O)[O-])cc12]

Compound 5 (0.4 g, 1.6 mmol) is suspended in nitrobenzene (40 mL). Bromine (140 μL, 2.5 mmol, 1.5 eq.) is added and the reaction mixture is stirred for 18 h at 120° C. After cooling to 0° C., the precipitated precipitate is filtered off and washed with diethyl ether. Further diethyl ether (100 mL) is added to the filtrate and the mixture is cooled to 0° C. for one hour. The precipitate formed is filtered off and washed with diethyl ether. The combined solids are dried in vacuo to give compound 6 (442 mg, 1.38 mmol, 86%) as a yellow solid.

MS (ESI): m/z (positive mode): 341.0 [M+Na]⁺, 660.9 [2M+Na]⁺, 978.9 [3M+Na]⁺,

HR-MS (ESI): C₁₃H₇N₂O₃: calc. [M+H]⁺: 318.9713, found: 318.9719, [M+Na]⁺: calc. 340.9532, found: 340.9539.

¹H-NMR (400 MHz, DMF-d₇): δ [ppm]=12.66 (s, 1H, NH), 9.08 (d, J=2.7 Hz, 1H, 1-H), 8.54 (dd, J=9.2, 2.7 Hz, 1H, 3-H), 8.39 (d, J=2.4 Hz, 1H, 8-H), 7.99 (dd, J=8.8, 2.4 Hz, 1H, 6-H), 7.81 (d, J=9.2 Hz, 1H, 4-H), 7.70 (d, J=8.8 Hz, 1H, 5-H),

¹³C-NMR (101 MHz, DMSO-d₄): δ [ppm]=176.8, 146.0, 142.7, 141.2, 138.2, 129.4, 128.8, 124.2, 123.7, 121.8, 120.8, 120.5, 116.3;

HPLC: t_{R}=12.32 min (ACN/H₂O+0.1% TFA; 30/70→100/0 in 12 min; detection at 254 nm)

### (IV) 7-[(3-hydroxypropyl)thio]-2-nitroacridin-9(10H)-one (7)

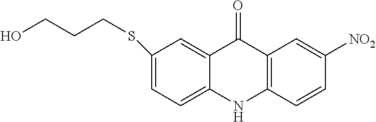 [SMILES: O=c1c2cc(SCCCO)ccc2[nH]c2ccc([N+](=O)[O-])cc12]

Compound 6 (500 mg, 1.57 mmol) is added to a suspension of K₂CO₃ (433 mg, 2 eq.) in DMF (40 ml). 3-Mercapto-1-propanol (216 mg, 2.35 mmol, 1.5 eq.) and triethylamine (150 μL) were added. Argon is passed through the solution for 10 min and then xantphos (308 mg, 530 μmol, 0.34 eq.) and Pd₂(dba)₃ (402 mg, 430 μmol, 0.28 eq.) are added. The reaction mixture is stirred for 23 h at 105° C. under argon atmosphere until complete conversion (HPLC). The solvent is evaporated off under reduced pressure, and the residue is dissolved in ACN (+10% TFA) and loaded onto a Celite. Flash chromatography (Reveleris (SiO₂) 40 g cartridge, ACN/DCM, ACN: 8%→66%) yields pure product 7 (229 mg, 0.69 mmol, 44%).

MS (ESI): m/z (positive mode): 331.1 [M+H]⁺, 353.1 [M+Na]⁺, 683.1 [2M+Na]⁺, 1113.2 [3M+Na]⁺.

HR-MS (ESI): C₁₆H₁₄N₂O₄S: calc. [M+H]⁺: 331.0747, found: 331.0751, [M+Na]⁺: calc. 353.0566, gefunden: 353.0566.

¹H-NMR (400 MHz, DMF-d₇): δ [ppm]=12.40 (s, 1H, NH), 8.95 (d, J=2.7 Hz, 1H, 1-H), 8.45 (dd, J=9.2, 2.7 Hz, 1H, 3-H), 8.08 (d, J=2.3 Hz, 1H, 8-H), 7.78 (dd, J=8.7, 2.3 Hz, 1H, 6-H), 7.65 (d, J=9.2 Hz, 1H, 4-H), 7.56 (d, J=8.7 Hz, 1H, 5-H), 3.52 (t, J=6.1 Hz, 2H, 3′-H), 3.13-3.01 (m, 2H, 1′-H), 1.83-1.66 (m, 2H, 2′-H).

¹³C-NMR (101 MHz, DMSO-d₆): δ [ppm]=176.2, 144.9, 141.4, 139.4, 135.6, 131.6, 127.4, 125.4, 123.3, 121.8, 119.9, 119.4, 109.9, 60.0, 32.5, 30.3.

HPLC: t_{R}=9.67 min (ACN/H₂O+0.1% TFA; 30/70→100/0 in 12 min; detection at 254 nm)

### (V) 7-[(3-Hydroxypropyl)sulfonyl]-2-nitroacridin-9(10H)-one (8)

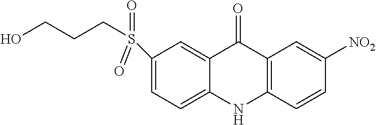 [SMILES: O=c1c2cc([N+](=O)[O-])ccc2[nH]c2ccc(S(=O)(=O)CCCO)cc12]

Compound 7 (255 mg, 0.77 mmol) is suspended in a mixture of acetic acid (10 mL) and water (2.4 mL). Na₂WO₄ 2H₂O (63 mg, 0.19 mmol, 0.25 eq) is added and the suspension is cooled down to 0° C. Hydrogen peroxide (10 mL, 50% (v/v)) is then added over 10 minutes. The suspension is warmed to room temperature and stirred for an additional 5 h with dissolution of the solid. After complete reaction of the reactant (HPLC), the reaction mixture is freeze-dried overnight. The crude product is dissolved in DMF (0.5 mL) and purified using a Biotage SNAP Ultra 25 g cartridge. Flash chromatography (ACN/DCM, ACN 10%→100%) results in 8 (179 mg, 0.49 mmol, 64%) as an orange solid.

MS (ESI): m/z (positive mode): 363.1 [M+H]⁺, 385.1 [M+Na]⁺, 747.1 [2M+Na]⁺, 1109.2 [3M+Na]⁺.

HR-MS (ESI): C₁₆H₁₄N₂O₆S: calc. [M+H]⁺: 363.0645, found: 363.0650, [M+Na]⁺: calc. 385.0465, found: 385.0465.

¹H-NMR (400 MHz, DMF-d₇): δ [ppm]=12.86 (s, 1H, NH), 9.06 (d, J=2.7 Hz, 1H, 1-H), 8.79 (d, J=2.1 Hz, 1H, 8-H), 8.58 (dd, J=9.2, 2.7 Hz, 1H, 3-H), 8.26 (dd, J=8.7, 2.1 Hz, 6-H), 7.89 (d, J=8.7 Hz, 1H, 5-H), 7.84 (d, J=9.2 Hz, 1H, 4-H), 4.74 (bs, 1H, OH), 3.60 (t, J=6.2 Hz, 2H, 1′-H), 3.56-3.41 (m, 2H, 3′-H), 1.93-1.81 (m, 2H, 2′-H).

¹³C-NMR (101 MHz, DMF-d₇): δ [ppm]=177.5, 146.1, 145.15, 143.2, 134.5, 133.6, 129.2, 128.7, 124.0, 121.6, 121.5, 120.8, 120.8, 60.4, 53.9, 27.6.

HPLC: t_{R}=7.68 min (ACN/H₂O+0.1% TFA; 30/704100/0 in 12 min; detection at 254 nm)

### (VI) 7-[(3-hydroxypropyl)sulfonyl]-2-aminoacridin-9(10H)-one (2)

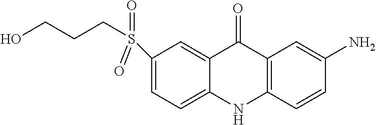 [SMILES: Nc1ccc2[nH]c3ccc(S(=O)(=O)CCCO)cc3c(=O)c2c1]

Pd/C (10%, in oxidized form, 8 mg) is suspended in 2-propanol (5 mL) in an argon atmosphere. Compound 8 (20 mg, 55 μmol) is added and a hydrogenate atmosphere is established. The suspension is stirred at room temperature overnight with partial dissolution of the solid. The yellow fluorescent solution is filtered over Celite to remove the Pd/C and the solid is washed with 2-propanol. The solvent is then removed under reduced pressure. The pure compound 2 is obtained (HPLC, 12 mg, 37 μmol, 68%).

MS (ESI): m/z (positive mode): 333.1 [M+H]⁺, 355.1 [M+Na]⁺, 665.2 [2M+H]⁺, 687.2 [2M+Na]⁺.

HR-MS (ESI): C₁₆H₁₆N₂O₄S: calc. [M+H]⁺: 333.0904, found: 333.0911, [M+Na]⁺: calc. 355.0723, found: 355.0730.

¹H-NMR (400 MHz, DMSO-d₄): δ [ppm]=12.03 (s, 1H, NH), 8.62 (d, J=2.2 Hz, 1H, 1-H), 7.98 (dd, J=8.8, 2.2 Hz, 1H, 3-H), 7.65 (d, J=8.8 Hz, 1H, 4-H), 7.46-7.37 (m, 2H, 6-H, 8-H), 7.19 (dd, J=8.8, 2.7 Hz, 1H, 5-H), 5.39 (s, 2H, NH₂), 4.61 (t, J=5.3 Hz, 1H, OH), 3.40 (q, J=5.9 Hz, 2H, 3′-H), 3.35-3.27 (m, 1H, 1′-H), 1.78-1.60 (m, 2H, 2′-H).

¹³C-NMR (101 MHz, DMSO-d₆): δ [ppm]=175.8, 144.6, 142.5, 132.4, 129.9, 129.4, 127.9, 123.8, 122.5, 118.7, 118.6, 117.9, 105.9, 58.7, 52.5, 26.2.

HPLC: t_{R}=4.2 min (ACN/H₂O+0.05 M TEAB pH˜8; 90/10→450/0 in 12 min; detection at 254 nm)

### (VII) 7-[[3-(O,O-di-tert-butylphosphate)propyl]sulfonyl]-2-nitroacridin-9(10H)-one (9)

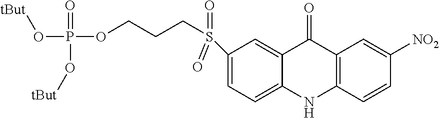

Compound 8 (58 mg, 162 μmol) is dissolved in DMF (1 mL) under argon atmosphere in a dry flask. 1H-tetrazole (39 mg, 0.561 mmol, 4 eq.) is dissolved in DMF (1 mL) and added together with di-tert-butyl N,N-diisopropylphosphoramidite (203 μL, d=0.88 g/mL, 179 mg, 0.646 mmol, 4 eq.). The reaction mixture is stirred at 40° C. for 1.5 h. After cooling to room temperature, mCPBA (139 mg, 0.805 mmol, 5 eq.) dissolved in DCM (1 mL) is added. The reaction mixture is stirred for 1 h and HPLC indicates complete conversion of the starting material. The solvents are removed under reduced pressure. The residue is dissolved in DMF (300 μL) and placed in a preparative HPLC (ACN/H₂O+0.1% TFA 40/60→70/30, in 20 min). Pure compound 9 (59 mg, 106 μmol, 65%) is obtained.

MS (ESI): m/z (positive mode): 577.1 [M+Na]⁺, 1131.3 [2M+Na]⁺.

HR-MS (ESI): C₂₄H₃₁N₂O₉S: calc. [M+H]⁺: 555.1561, found: 555.1565, [M+Na]⁺: calc. 577.1380, found: 577.1380.

¹H-NMR (400 MHz, DMF-d₇): δ [ppm]=12.95 (s, 1H, NH), 9.05 (d, J=2.7 Hz, 1H, 1-H), 8.79 (d, J=2.2 Hz, 1H, 8-H), 8.57 (dd, J=9.2, 2.7 Hz, 1H, 3-H), 8.27 (dd, J=8.8, 2.2 Hz, 1H, 6-H), 7.90 (d, J=8.8 Hz, 1H, 5-H), 7.85 (d, J=9.2 Hz, 1H, 4-H), 4.06 (m, 2H, 3′-H), 3.60-3.41 (m, 2H, 1′-H), 2.12-2.00 (m, 2H, 2′-H), 1.41 (s, 18H, ^{t}Bu).

¹³C-NMR (101 MHz, DMF-d₇): δ [ppm]=177.2, 145.9, 145.0, 143.0, 133.8, 133.3, 129.0, 128.6, 123.7, 121.4, 121.3, 120.7, 120.5, 82.5, 82.5, 65.4, 53.1, 30.0, 30.0, 25.1.

³¹P-NMR (162 MHz, DMF-d₇) δ [ppm]=−9.78.

HPLC: t_{R}=11.62 min (ACN/H₂O+0.1% TFA; 30/704100/0 in 12 min; detection at 254 nm)

### (IIX) 7-[(3-phosphopropyl)sulfonyl]-2-aminoacridin-9(10H)-one (1)

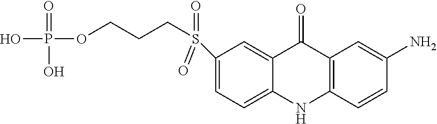 [SMILES: Nc1ccc2[nH]c3ccc(S(=O)(=O)CCCOP(=O)(O)O)cc3c(=O)c2c1]

Pd/C (10%, in oxidized form, 11 mg) is suspended in 2-propanol (3 mL) in an argon atmosphere. Compound 9 (24 mg, 43 μmol) is added and a hydrogenate atmosphere is established. The suspension is stirred at room temperature for 21 h with partial dissolution of the solid. The solution is filtered over Celite to remove the Pd/C and the solvent is evaporated off. HPLC indicates complete conversion of the reactant. The crude product is suspended in water (5 ml) and cooled to 0° C. TFA (10 mL) is then added dropwise. Warm the reaction mixture to room temperature and stir for 3 h. HPLC shows complete deprotection of the phosphate. The reaction mixture is freeze-dried overnight and then dissolved in aq. TEAB buffer (pH 8). The crude product is purified via preparative HPLC (ACN/H₂O (0.05% v/v TEAB buffer pH 8, 5/95%→27/73%, in 10 min, t_{R}=8.3 min). The triethylammonium salt (1.5 eq.) of compound 1 is obtained as a brown solid (13 mg, 31 μmol, 86%).

MS (ESI): m/z (positive mode): 413.1 [M+H]⁺, 435.0 (M+H)⁺, 457.0 [M+2Na]⁺,

HR-MS (ESI): C₁₆H₁₇N₂O₇PS: calc. [M+H]⁺: 413.0572, found: 413.0567, [M+Na]⁺: calc. 435.0386, found: 435.0393.

¹H-NMR (400 MHz, D₂O): δ [ppm]=8.37 (d, J=2.1 Hz, 1H, 1-H), 7.83 (dd, J=8.9, 2.1 Hz, 1H, 3-H), 7.21 (d, J=8.9 Hz, 1H, 4-H), 7.06-6.95 (m, 2H, 6-H, 8-H), 6.91 (d, J=8.7 Hz, 1H, 5-H), 3.82 (q, J=5.9 Hz, 2H, 1′-H), 3.65-3.42 (m, 2H, 3′-H), 3.15 (q, J=7.4 Hz, 10H, NEt₃) 2.08-1.95 (m, 2H, 2′-H), 1.24 (t, J=7.4 Hz 0.15H, NEt₃),

¹³C-NMR (100 MHz, D₂O): δ [ppm]=177.3, 142.2, 141.9, 133.9, 129.7, 128.7, 127.9, 126.1, 120.7, 118.9, 118.6, 117.3, 107.8, 61.9, 52.9, 46.6 (NEt₃), 23.9, 8.2 (NEt₃).

³¹P-NMR (162 MHz, D₂O): δ [ppm]=3.76.

### (IX) Synthesis of the Glucose-Conjugates

Synthesis of the Compound 1-GLU

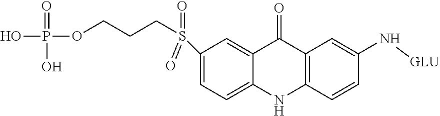

Compound 17 (3 mg, 7 μmol) is dissolved in a malonic acid solution (0.36 mL, 1 M in DMSO). Aqueous glucose solution (72 μL, 0.1 M, 1.1 eq.) and pic-BH₃ (87 μL, 1 M, 12 eq., in DMSO) are added. The reaction mixture is incubated at 35° C. for 18 h on a shaker. After addition of H₂O/ACN (3:1), the reaction mixture is freeze-dried. The crude product is purified via preparative HPLC (ACN/H₂O+0.1% TFA: 2/98→30/70, in 10 min). Pure 1-Glu is obtained as an orange solid (2.1 mg, 3 μmol, 33%).

MS (ESI): m/z (negative mode)=575.1 [M]⁻

Synthesis of the Compound 2-GLU

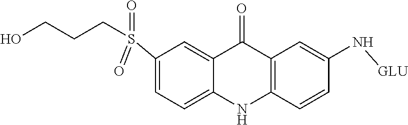

Compound 2 (3.35 mg, 10 μmol) is dissolved in a malonic acid solution (0.4 mL, 1 M in DMSO). Aqueous glucose solution (90 μL, 0.1 M, 1.1 eq.) and pic-BH₃ (108 μL, 1 M, 12 eq., in DMSO) are added. The reaction mixture is incubated at 35° C. for 18 h on a shaker. After addition of H₂O/ACN (3:1), the reaction mixture is freeze-dried. The crude product is purified via preparative HPLC (ACN/H₂O+0.1% TFA: 2/98→30/70, in 10 min). Pure 2-Glu is obtained as an orange solid (2 mg, 4 μmol, 40%).

MS (ESI): m/z (negative mode)=495.2 [M]⁻

HR-MS (ESI): C₂₂H₂₈N₂O₉S: calc. [M+H]⁺: 497.1600, found: 497.1588.

Photophysical Properties of Fluorescent Markers

The spectroscopic properties are shown by way of example in the following two tables using compounds 1 and 2.

For compound 1, the following properties are obtained as a function of the solvent:

TABLE 1

| | 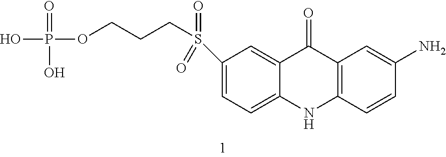 [SMILES: Nc1ccc2[nH]c3ccc(S(=O)(=O)CCCOP(=O)(O)O)cc3c(=O)c2c1] | |
|---|---|---|
| Solvent | MEOH | TEAB (pH 8) |
| Absorption, nm (ε, M⁻¹ · cm⁻¹) | 428 (2530) | 423 (2750) |
| Emission (nm) | 567 | 583 |
| Absolute quantum yield φ f, % | 11.5 (420 nm) | 5.3 (420 nm) |
| (Excitation wavelength) | | |
| Lifetime (τ, ns) | 10.1 | 3.1 |
| Stokes-Shift (nm) | 139 | 157 |
| Stokes-Shift (10³ cm⁻¹) | 73 000 | 64 000 |

The fluorescence marker is characterized by high quantum yields even in different solvents and shows emission at wavelengths that differ from the wavelengths of conventional fluorescence markers, especially in glycan analysis. It is therefore possible to work in parallel with two different fluorescence markers. Thus, sample and reference can be labeled differently and simultaneously detected in different wavelength ranges. In this way, a pre- or post-run with a separate molecular weight reference can be omitted.

Compound 1 can also be characterized by the following parameters:

MS(ESI): m/z (positive mode): 413.1 [M+H]⁺, 435.0 (M+H)⁺, 457.0 [M+2Na]⁺

HR-MS (ESI): C16H17N2O7PS: calc. [M+H]⁺: 413.0572, found: 413.0567, [M+Na]⁺: calc. 435.0386, found: 435.0393.

¹H-NMR (400 MHz, D₂O): δ [ppm]=8.37 (d, J=2.1 Hz, ¹H, 1-H), 7.83 (dd, J=8.9, 2.1 Hz, ¹H, 3-H), 7.21 (d, J=8.9 Hz, ¹H, 4-H), 7.06-6.95 (m, 2H, 6-H, 8-H), 6.91 (d, J=8.7 Hz, ¹H, 5-H), 3.82 (q, J=5.9 Hz, 2H, 1′-H), 3.65-3.42 (m, 2H, 3′-H), 3.15 (q, J=7.4 Hz, 10H, NEt₃) 2.08-1.95 (m, 2H, 2′-H), 1.24 (t, J=7.4 Hz 0.15H, NEt₃).

¹³C-NMR (100 MHz, D₂O): δ [ppm]=177.3, 142.2, 141.9, 133.9, 129.7, 128.7, 127.9, 126.1, 120.7, 118.9, 118.6, 117.3, 107.8, 61.9, 52.9, 46.6 (NEt₃), 23.9, 8.2 (NEt₃).

³¹P-NMR (162 MHz, D₂O): δ [ppm]=3.76.

For compound 2, the following properties are obtained as a function of the solvent:

TABLE 2

| | 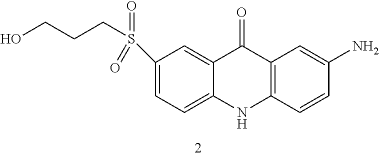 | |
|---|---|---|
| Solvent | MEOH | TEAB (pH 8) |
| Absorption, nm (ε, M⁻¹ · cm⁻¹) | 427 (2200) | 423 (2940) |
| Emission (nm) | 567 | 588 |
| Absolute quantum yield φ f, % | 17 (420) | 5.3 (4520) |
| (Excitation wavelength) | | |
| Lifetime (τ, ns) | 9.7 | 2.8 |
| Stokes-Shift (nm) | 138 | 165 |
| Stokes-Shift (10³ cm⁻¹) | 72 000 | 61 000 |

The fluorescent marker is characterized by high quantum yields in different solvents and shows emission at wavelengths that differ from the wavelengths of conventional fluorescent markers, especially in glycan analysis. It is therefore possible to work in parallel with two different fluorescence markers. The sample and reference can thus be labeled differently and detected simultaneously in different wavelength ranges. In this way, a pre- or post-run with a separate molecular weight reference can be omitted.

Compound 2 can also be characterized by the following parameters:

MS (ESI): m/z (positive mode): 333.1 [M+H]⁺, 355.1 [M+Na]⁺, 665.2 [2M+H]⁺, 687.2 [2M+Na]⁺.

HR-MS (ESI): C16H16N2O4S: calc. [M+H]⁺: 333.0904, found: 333.0911, [M+Na]⁺: calc. 355.0723, found: 355.0730.

¹H-NMR (400 MHz, DMSO-d 6): δ [ppm]=12.03 (s, 1H, NH), 8.62 (d, J=2.2 Hz, 1H, 1-H), 7.98 (dd, J=8.8, 2.2 Hz, 1H, 3-H), 7.65 (d, J=8.8 Hz, 1H, 4-H), 7.46-7.37 (m, 2H, 6-H, 8-H), 7.19 (dd, J=8.8, 2.7 Hz, 1H, 5-H), 5.39 (s, 2H, NH 2), 4.61 (t, J=5.3 Hz, 1H, OH), 3.40 (q, J=5.9 Hz, 2H, 3′-H), 3.35-3.27 (m, 1H, 1′-H), 1.78-1.60 (m, 2H, 2′-H),

¹³C-NMR (101 MHz, DMSO-d₆): δ [ppm]=175.8, 144.6, 142.5, 132.4, 129.9, 129.4, 127.9, 123.8, 122.5, 118.7, 118.6, 117.9, 105.9, 58.7, 52.5, 26.2.

HPLC: tr=4.2 min (ACN/H₂O+0.05 M TEAB pH˜ 8; 90/10→50/0 in 12 min; detection at 254 nm)

Photophysical Properties of Fluorescent Marker Conjugates

The spectroscopic properties of the fluorescent marker conjugates with a glucose molecule are shown as examples in the following two tables.

The following properties are obtained for the conjugate of compound 1:

TABLE 3

| | 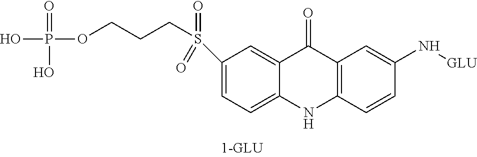 |
|---|---|
| Solvent | TEAB (pH 8) |
| Absorption, nm | 429 (2750) |
| (ε, M⁻¹ · cm⁻¹) | |
| Emission (nm) | 593 |
| Absolute quantum yield φ f, | 5.3 (430 nm) |
| % (Excitation wavelength) | |
| Lifetime (τ, ns) | 3.4 |
| Stokes-Shift (nm) | 164 |
| Stokes-Shift (10³ cm⁻¹) | 61 000 |

The fluorescent marker is characterized by high quantum yields in different solvents and shows emission at wavelengths that differ from the wavelengths of conventional fluorescent markers, especially in glycan analysis. It is therefore possible to work in parallel with two different fluorescence markers. The sample and reference can thus be labeled differently and detected simultaneously in different wavelength ranges. In this way, a pre- or post-run with a separate molecular weight reference can be omitted. It should also be noted that the spectroscopic properties of the fluorescent label change only slightly due to conjugate formation. This is an indication of a stable spectroscopic system.

The following properties are obtained for the conjugate of compound 2:

TABLE 4

| | 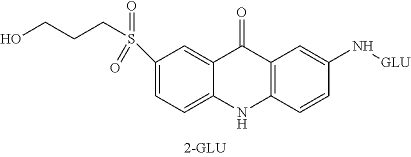 |
|---|---|
| Solvent | TEAB (pH 8) |
| Absorption, nm (ε, M⁻¹ · cm⁻¹) | 433 (2490) |
| Emission (nm) | 597 |
| Absolute quantum yield φ f, % | 4.5 (430 nm) |
| (excl. wavelength) | |
| lifetime (τ, ns) | 3.4 |
| Stokes-Shift (nm) | 164 |
| Stokes-Shift (10³ cm⁻¹) | 61 000 |

The fluorescent marker is characterized by high quantum yields in different solvents and shows emission at wavelengths that differ from the wavelengths of conventional fluorescent markers, especially in glycan analysis. It is therefore possible to work in parallel with two different fluorescence markers. The sample and reference can thus be labeled differently and detected simultaneously in different wavelength ranges. In this way, a pre- or post-run with a separate molecular weight reference can be omitted. It should also be noted that the spectroscopic properties of the fluorescent label change only slightly due to conjugate formation. This is an indication of a stable spectroscopic system.

The foregoing description of the embodiments has been provided for purposes of illustration and description. It is not intended to be exhaustive or to limit the disclosure. Individual elements or features of a particular embodiment are generally not limited to that particular embodiment, but, where applicable, are inter-changeable and can be used in a selected embodiment, even if not specifically shown or described. The same may also be varied in many ways. Such variations are to be regarded as a departure from the disclosure, and all such modifications are intended to be included within the scope of the disclosure.

1. A fluorescent marker according to the following formula I 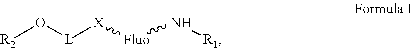
wherein
R₁=—H or C1-C6 Alkyl;
R₂=—H or —PO(OH)₂
X=—SO₂— or —PO(OH)—;
L=—(CH₂)ₙ—CHR₃—CH₂—, n=1, 2, 3 and R₃=—H or —(CH₂)—OPO(OH)₂;
wherein Fluo is selected from the group consisting of substituted or non-substituted C10-C30 acridones, aminoacridines or pyrenes.

2. The fluorescent marker according to claim 1, wherein Fluo is selected from the group consisting of the fluorophores according to the formula II-IV 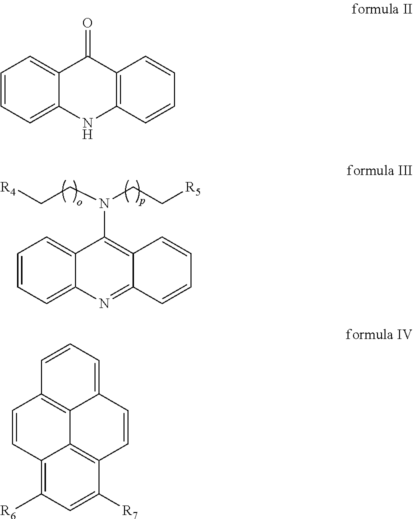
wherein
R₄, R₅=each independently of one another —OPO(OH)₂ or —OH;
R₆, R₇=each independently of one another —SO₂—CH₂—CHR₂—(CH₂)_{q}—R₄ or —PO(OH)CH₂—CHR₂—(CH₂)_{q}—R₄ and q=1, 2, 3;
o, p=each independently of one another 1, 2, 3;
and the Fluo according to the formula II-IV can be linked at each possible binding site to NH and X.

3. The fluorescent marker according to claim 1, wherein the fluorophore Fluo corresponds to the formula V-VII 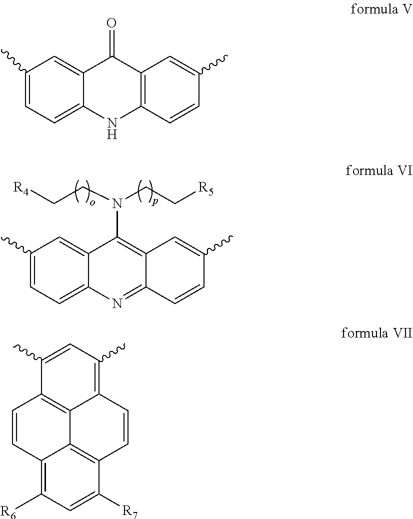
wherein the fluorophore can be linked via the curved bonds to NH and X.

4. The fluorescent marker according to claim 1, wherein X=—SO₂—.

5. The fluorescent marker according to claim 1, wherein in the linker L R₃=—(CH₂)—OPO(OH)₂.

6. The fluorescent marker according to claim 1, wherein in the fluorophore according to the formula III or VI R₄ and R₅ each are —OPO(OH)₂.

7. The fluorescent marker according to claim 1, wherein in the Fluorophore according to the formula IV and VII R₆ and R₇ are each —PO(OH)—CH₂—CHR₂—(CH₂)—OPO(OH)₂.

8. A use of a fluorescent marker according to claim 1 as a standard in a glycan analysis.

9. A conjugate of a fluorescent marker and a glycan, wherein
the Glycan is covalently bound to at least one fluorescent marker according to claim 1.

10. The conjugate according to claim 9, wherein the glycan is selected from the group consisting of mono-, oligo-, polysaccharides, glycoproteins, glycolipids, proteoglycans or mixtures thereof.

11. A kit-of-parts at least comprising
one or more container comprising one or more solvents and
one or more container each containing a fluorescence marker according to claim 1 or conjugates according to claim 9.
